(19) United States
(12) Patent Application Publication
(10) Pub. No.: US 2026/0069465 A1
(43) Pub. Date: Mar. 12, 2026

(54) APPARATUS AND METHOD FOR PRODUCING ABSORBENT BED PADS OR ABSORBENT PET MATS

(71) Applicant: GDM S.p.A, Bologna (IT)

(72) Inventor: Alessandro ZAVALLONI, Bologna (IT)

(21) Appl. No.: 19/284,771

(22) Filed: Jul. 30, 2025

(30) Foreign Application Priority Data
Sep. 4, 2024 (IT) 10202400019675

(51) Int. Cl. A61F 13/15; A61F 13/53

(52) U.S. Cl. CPC A61F 13/15642; A61F 13/15747; A61F 2013/15056; A61F 2013/15186; A61F 2013/530226; A61F 2013/530343; A61F 2013/530481

(57) ABSTRACT

An apparatus for producing absorbent bed pads, or absorbent pet mats, having an absorbent core with a support layer and absorbent fibres arranged on a first surface of the support layer. The apparatus includes, in sequence along an advancing direction of a support layer strip, a preparation station, a deposition station downstream of the preparation station and a forming station downstream of the deposition station. The preparation station includes first folding members configured to fold opposite longitudinal flaps of the support layer strip over a service surface of the support layer strip. The deposition station includes a deposition device configured to deposit the absorbent fibres on a deposition surface of the support layer strip opposite the service surface. The forming station includes second folding members configured to fold the longitudinal flaps of the support layer strip over the deposition surface. A method that can be carried out by the apparatus.

### FIELD OF THE INVENTION

The present invention refers to an apparatus and a method for producing absorbent bed pads or absorbent pet mats.

### BACKGROUND

Absorbent pads act as a barrier between a user and the bed or other surfaces on which the user stands. Similarly, the absorbent mats act as a barrier between the pet and the litter box or other surfaces on which the pet stands. In the following description reference will be made to the non-limiting example of absorbent bed pads, but what will be described also applies to the absorbent mats.

The absorbent bed pads are articles of assistance for the management of the incontinence. They are designed to absorb any leakage of urine or other body secretions during sleep, keeping the bed dry and comfortable.

Typically, an absorbent bed pad has a substantially rectangular shape and comprises a lower layer (also called “backsheet”), an upper layer (also called “topsheet”) and an absorbent core arranged between the lower layer and the upper layer.

The lower layer is configured to be arranged on the bed and is made of a material impermeable to the body fluids, for example polyethylene or bioplastic.

The upper layer is configured to be arranged in a position contiguous to the user's body and is made of a material that is permeable to the body fluids. This layer is typically made of a soft, breathable material and allows the body fluid to pass through this layer and reach the absorbent core below, keeping the user dry and reducing the risk of irritation.

The absorbent core comprises a support layer and a plurality of absorbent fibres arranged on a surface of the support layer. The absorbent core has the function of absorbing the body fluids that pass through the upper layer and retaining them without leakage even for several hours.

The support layer is generally made of paper.

Typically, the absorbent fibres comprise cellulose and/or one or more super-absorbent polymers (SAP), for example sodium polyacrylate, typically in granular form, which may be confined inside, or within a coating of, cellulose or other similar materials.

The realization of the absorbent bed pads envisages, among other things, supplying a support layer strip onto a conveyor belt and depositing the absorbent fibres on a deposition surface of the support layer strip, over the entire width of the latter.

The deposition of the absorbent fibres takes place by fall from a deposition device arranged above the conveyor belt. The fall of the absorbent fibres is aided by an air flow which, coming from the deposition device, passes through the support layer strip and the conveyor belt.

The deposition device typically has a width greater than that of the support layer strip, so as to be able to deposit the absorbent fibres over the entire width of the latter regardless of the size of the absorbent bed pad that it is wished to be made. The excess absorbent fibres along the width of the support layer strip are collected.

In order to separate in a precise manner the absorbent fibres deposited on the support layer strip from those in excess, it is known to provide, at a downstream end portion of the conveyor belt with reference to its movement direction and along a transverse direction with respect to the aforesaid movement direction, two air blowers, each arranged near a respective side of the conveyor belt and, therefore, at a respective longitudinal edge of the support layer strip deposited on the conveyor belt. The air blowers are configured to each generate a respective concentrated air jet that laps the respective longitudinal edge of the support layer strip, thereby forcing the separation of the absorbent fibres in excess from those deposited on the support layer strip.

### SUMMARY

The Applicant has observed that there is a need to realize types of absorbent bed pads provided with folds at the opposite longitudinal edges.

The Applicant is aware of a method for realizing such types of absorbent pads which provides, after the deposition of the absorbent fibres over the deposition surface of the support layer strip, the arrangement on the support layer strip of a containment layer strip having a width greater than that of the support layer strip and the folding of the opposite longitudinal flaps of the containment layer strip on a service surface of the support layer strip opposite the deposition surface.

However, the Applicant has observed that this realization method requires the use of a further strip (precisely the containment layer strip), with a consequent increase in production cost.

The Applicant has therefore thought of realizing the aforesaid folds by folding the longitudinal flaps of the support layer strip over the deposition surface.

However, the Applicant has observed that in order to be able to fold the longitudinal flaps of the support layer strip, it is necessary to use a support layer strip having a width greater than that of the portion of the deposition surface on which the absorbent fibres are deposited. In this case, it would not be possible to achieve the desired precise separation of the excess absorbent fibres from those deposited over the deposition surface by the aforesaid air blowers since the concentrated air jets emitted by these air blowers would impact on the opposite longitudinal flaps of the support layer strip, which would therefore hinder the air flow emitted by the aforesaid air blowers.

The Applicant has thought about how to overcome this drawback and has realized that it is possible to do so by first carrying out a fold of the longitudinal flaps of the support layer strip over the service surface of the support layer strip (and therefore on the opposite side with respect to the deposition surface), so as to hide these longitudinal flaps below the support layer strip, then the deposition of the absorbent fibres over the deposition surface and the subsequent separation by the aforesaid air blowers of the absorbent fibres in excess from those deposited (without therefore the longitudinal flaps of the support layer strip being in the way of this separation), and finally the fold of the longitudinal flaps of the support layer strip over the deposition surface.

The present invention therefore concerns, in a first aspect thereof, an apparatus for producing absorbent bed pads, or absorbent pet mats, of the type comprising an absorbent core comprising a support layer and a plurality of absorbent fibres arranged on a first surface of the support layer.

Preferably, a preparation station is provided.

Preferably, the preparation station comprises first folding members.

Preferably, the first folding members are configured to fold opposite longitudinal flaps of a support layer strip over a service surface of the support layer strip.

Preferably, a deposition station is provided.

Preferably, the deposition station is arranged downstream of the preparation station along an advancing direction of the support layer strip.

Preferably, the deposition station comprises a deposition device.

Preferably, the deposition device is configured to deposit the plurality of absorbent fibres on a deposition surface of the support layer strip opposite the service surface.

Preferably, a forming station is provided.

Preferably, the forming station is arranged downstream of the deposition station along the advancing direction.

Preferably, the forming station comprises second folding members configured to fold the opposite longitudinal flaps of the support layer strip over the deposition surface.

The present invention allows to separate with precision, by means of the aforesaid air blowers, the excess absorbent fibres from those deposited over the deposition surface of the support layer strip. During this separation the concentrated air jets emitted by the blowers do not impact on the longitudinal flaps of the support layer strip since they have been previously folded over the service surface by the first folding members. Only after the aforesaid separation has taken place the longitudinal flaps of the support layer strip are folded over the deposition surface by the second folding members, obtaining the desired folds.

In the following, the position assumed by the longitudinal flaps of the support layer strip before they are folded over the service surface is referred to as the “initial position”, that assumed by the longitudinal flaps of the support layer strip after they are folded over the service surface and before they are folded over the deposition surface is referred to as the “intermediate position” and that assumed by the longitudinal flaps of the support layer strip after they are folded over the deposition surface is referred to as the “final position”.

Thus, the first folding members move the longitudinal flaps of the support layer strip from the initial position to the intermediate position and the second folding members move the longitudinal flaps of the support layer strip from the intermediate position to the final position.

When the longitudinal flaps of the support layer strip are in their final position, a part of the absorbent fibres deposited over the deposition surface is located interposed between the deposition surface and a respective folded longitudinal flap.

In a second aspect thereof, the present invention concerns a method for producing absorbent bed pads, or absorbent pet mats, of the type comprising an absorbent core comprising a support layer and a plurality of absorbent fibres arranged on a first surface of the support layer.

Preferably, opposite longitudinal flaps of a support layer strip are folded over a service surface of the support layer strip.

Preferably, it is provided for depositing the plurality of absorbent fibres on a deposition surface of the support layer strip opposite the service surface. This deposition takes place after folding the opposite longitudinal flaps over the service surface.

Preferably, it is provided for folding the opposite longitudinal flaps of the support layer strip over the deposition surface. This fold occurs subsequent to the deposition of the plurality of absorbent fibres over the deposition surface of the support layer strip.

The method of the invention can be carried out by employing an apparatus according to the first aspect of the present invention and allows to achieve the same advantages discussed with reference to the apparatus of the invention.

The present invention may have, in both aspects discussed above, at least one of the preferred features described below. These features can therefore be present individually or in combination with each other, unless expressly stated otherwise, both in the apparatus of the first aspect of the present invention and in the method of the second aspect of the present invention.

Preferably, the deposition station comprises a pair of air blowers arranged downstream of the deposition device, at opposite sides with respect to the advancing direction.

The air blowers perform an air jet cut that allows to clearly separate the absorbent fibres deposited over the deposition surface from the others (here referred to as excess fibres), for the benefit of the quality of the produced absorbent pads.

Preferably, the air blowers are movable along a transverse direction orthogonal to the advancing direction, so as to be able to make absorbent bed pads of different sizes.

Preferably, the air blowers are arranged upstream of a downstream end portion of the conveyor belt along the advancing direction. The excess fibres are then separated from the others and recovered before the support layer strip leaves the conveyor belt.

Preferably, the second folding members are configured to move the longitudinal flaps of the support layer strip first from the intermediate position to the initial position and immediately after from the initial position to the final position.

Preferably, the first folding members are configured to continuously fold the opposite longitudinal flaps of the support layer strip over the service surface while the support layer strip passes through the preparation station.

Preferably, the first folding members are arranged at opposite sides with respect to the support layer strip when the support layer strip passes through the preparation station.

Preferably, the first folding members comprise first folding portions/elements arranged on one side with respect to the support layer strip and second folding portions/elements arranged on the opposite side with respect to the support layer strip.

The first and second folding portions/elements are preferably shaped and oriented in such a way as to interfere with the longitudinal flaps of the support layer strip during the movement of the support layer strip in the preparation station and fold them until reaching their intermediate position.

Preferably, the first folding members are movable along a transverse direction with respect to a first advancing path of the support layer strip in the preparation station. This movement makes it possible to realize absorbent bed pads of different sizes. In particular, it is possible to set the mutual distance between the first folding portions/elements and the second folding portions/elements as a function of the desired deposition surface width.

Preferably, the second folding members are configured to continuously fold the opposite longitudinal flaps of the support layer strip over the deposition surface while the support layer strip passes through the forming station.

Preferably, the second folding members are arranged at opposite sides with respect to the support layer strip when the support layer strip passes through the forming station.

Preferably, the second folding members comprise first folding portions/elements arranged on one side with respect to the support layer strip and second folding portions/elements arranged on the opposite side with respect to the support layer strip. The same applies as above with reference to the first folding members.

The first and second folding portions/elements preferably are shaped and oriented in such a way as to interfere with the longitudinal flaps of the support layer strip during the movement of the support layer strip along the advancing direction and fold them until first reaching their initial position and then their final position.

Preferably, the second folding members are movable along a transverse direction with respect to a second advancing path of the support layer strip in the forming station, so as to be able to make absorbent bed pads of different sizes. The same applies as above with reference to the first folding members.

Preferably, the preparation station comprises perforating members configured to perforate the support layer strip along its entire width while the support layer strip passes through the preparation station.

The Applicant has found that the support layer strip generates a resistance to the passage of the air flow and, since the paper of which the support layer strip is typically formed has not a homogeneous structure, this resistance is not uniform along the width of the support layer strip. This would cause an uneven distribution of the absorbent fibres on the support layer strip, resulting in adverse effects in terms of the quality of the produced absorbent pads. Thanks to the provision of the aforesaid perforating members the air flow coming from the deposition device substantially uniformly passes through the support layer strip along its entire width during the deposition of the absorbent fibres, allowing a homogeneous distribution of the absorbent fibres on the deposition surface of the support layer strip. This benefits the quality of the produced absorbent pads.

In a first preferred embodiment, the perforating members comprise a first roller configured to make a plurality of first holes on the entire support layer strip. These holes allow the resistance offered by the support layer strip to the air flow coming from the deposition device during the deposition of the absorbent fibres to be uniform over the entire deposition surface.

The first roller can be arranged upstream or downstream of the first folding members along the first advancing path.

Preferably, the first roller is arranged downstream of the first folding members along the first advancing path.

Preferably, the perforating members further comprise a pair of second rollers configured to each make a plurality of respective second holes at a respective longitudinal flap of the support layer strip folded over the service surface. The second rollers then make an additional perforation on the support layer strip only at the longitudinal flaps folded over the service surface, where then the support layer strip has two overlapping layers. This expedient makes it possible to uniform, along the entire width of the support layer strip, the resistance offered by this strip to the air flow coming from the deposition device during the deposition of the absorbent fibres, contributing to the achievement of a more homogeneous distribution of the absorbent fibres on the support layer strip.

Preferably, the pair of second rollers is arranged downstream of the first folding members along the first advancing path.

The pair of second rollers can be arranged upstream or downstream of the first roller along the first advancing path.

Preferably, the second rollers are movable along a transverse direction with respect to the first advancing path, so that they can be arranged at the folded longitudinal flaps of the support layer strip when the support layer strip passes through the preparation station.

In a second preferred embodiment, the perforating members comprise a roller comprising a central portion configured to make a plurality of first holes on a central longitudinal portion of the support layer strip.

In this case, preferably, said roller further comprises opposite end portions configured to each make a plurality of respective second holes at a respective longitudinal flap of the support layer strip folded over the service surface.

Preferably, the aforesaid roller is arranged downstream of the first folding members along the first advancing path.

Preferably, in both aforesaid preferred embodiments, the plurality of respective second holes differs from the plurality of first holes in at least one of the following parameters: size of the holes and number of holes for the same area.

Preferably, the opposite longitudinal flaps of the support layer strip are continuously folded over the service surface of the support layer strip while the support layer strip is moved along a first advancing path.

Preferably, the opposite longitudinal flaps of the support layer strip are continuously folded over the deposition surface of the support layer strip while the support layer strip is moved along a second advancing path.

Preferably the longitudinal flaps of the support layer strip are first moved from the intermediate position to the initial position and immediately after moved from the initial position to the final position. These two movements can be seamlessly carried out.

Preferably, prior to depositing the plurality of absorbent fibres on the deposition surface, the support layer strip is perforated along its entire width.

In a first preferred embodiment, perforating the support layer strip comprises making a plurality of first holes on the entire support layer strip.

Preferably, perforating the support layer strip further comprises, before making or after having made the plurality of first holes, making a plurality of second holes at each longitudinal flap of the support layer strip folded over the service surface.

In a second preferred embodiment, perforating the support layer strip comprises making a plurality of first holes on a central longitudinal portion of the support layer strip.

In this case, preferably, perforating the support layer strip further comprises making, simultaneously with the first holes, a plurality of second holes at opposite longitudinal flaps of the support layer strip.

Preferably, in both aforesaid preferred embodiments, the plurality of respective second holes differs from the plurality of first holes in at least one of the following parameters: size of the holes and number of holes for the same area.

### BRIEF DESCRIPTION OF THE DRAWINGS

Further characteristics and advantages of the present invention will become clearer from the following detailed description of preferred embodiments thereof, made with reference to the appended drawings and provided by way of indicative and non-limiting example, in which:

FIG. 1 is a schematic cross-sectional view of a support layer strip used to produce absorbent bed pads by an apparatus in accordance with the present invention, such as that of FIG. 7, this support layer strip being in an initial operating configuration thereof;

FIG. 2 is a schematic cross-sectional view of the support layer strip of FIG. 1 in a second intermediate operating configuration thereof;

FIG. 3 is a schematic cross-sectional view of a portion of the apparatus of FIG. 7 with the support layer strip in the intermediate operating configuration;

FIG. 4 is a schematic cross-sectional view of the support layer strip brought back to its initial operating configuration after absorbent fibres have been deposited thereon;

FIG. 5 is a schematic cross-sectional view of the support layer strip of FIG. 1 in a final operating configuration thereof after the absorbent fibres have been deposited thereon;

FIG. 6 is a schematic cross-sectional view of an absorbent bed pad comprising the support layer strip in its final operating configuration;

FIG. 7 is a schematic side elevational view of an apparatus in accordance with the present invention;

FIG. 8 is a schematic side elevation view on an enlarged scale of a first embodiment of a station of the apparatus of FIG. 7;

FIG. 9 is a schematic plan view of some members present in the station of FIG. 8;

FIG. 10 is a schematic plan view of a portion of the support layer strip when it is located at the plane X of FIG. 8;

FIG. 11 is a schematic plan view of a portion of the support layer strip when it is located at the plane XI of FIG. 8;

FIG. 12 is a schematic side elevation view on an enlarged scale of a second embodiment of the station of the apparatus of FIG. 7, alternative to that of FIG. 8;

FIG. 13 is a schematic plan view of a member present in the station of FIG. 12;

FIG. 14 is a schematic plan view of a portion of the support layer strip when it is located at the plane XIV of FIG. 12;

FIG. 15 is a schematic perspective view on an enlarged scale of a further member of the apparatus of FIG. 7.

### DETAILED DESCRIPTION

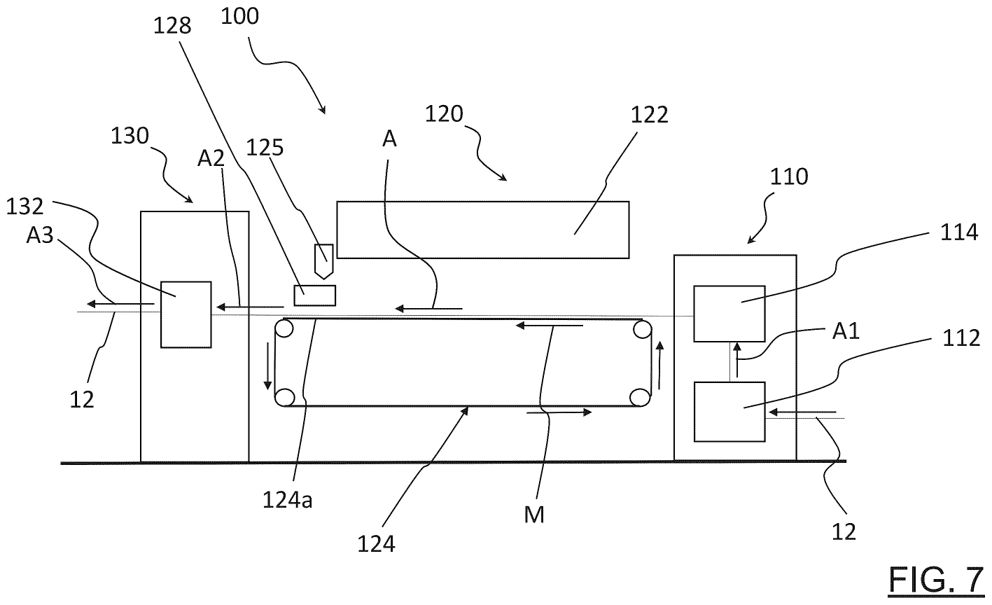

In FIG. 7, the reference numeral 100 is used to indicate an apparatus for producing absorbent bed pads in accordance with the present invention.

Without for this reason losing in generality, explicit reference will be made below to an absorbent pad, such as the one illustrated in FIG. 6 and indicated with reference numeral 2. In FIG. 6 the absorbent pad 2 is voluntarily illustrated with the relative components spaced apart from each other, so as to be able to clearly distinguish them. In reality these components are in contact with each other.

The absorbent pad 2 has a substantially rectangular shape and comprises a lower layer 3 (also called “backsheet”), an upper layer 4 (also called “topsheet”) superimposed on the lower layer 3 and an absorbent core 10a arranged between the lower layer 3 and the upper layer 4.

In the following of the present description and in the following claims, the terms “upper”, “lower”, “above”, “below” and the like are used referring to the position assumed by the absorbent pad 2 when it is arranged on a bed (not illustrated), with the lower layer 3 in contact with the bed and the upper layer 4 arranged in such a way as to come into contact with the body of the user.

The lower layer 3 is made of a material impermeable to the body fluids, for example polyethylene, polypropylene or bioplastic, while the upper layer 4 is made of a material permeable to the body fluids, for example non-woven fabric.

The absorbent core 10a comprises a support layer 12a and a plurality of absorbent fibres 20 arranged on a surface 13a of the support layer 12a. The surface 13a faces the upper layer 4.

The support layer 12a may for example be made of paper, while the absorbent fibres 20 comprise cellulose and/or one or more super-absorbent polymers (SAP), for example sodium polyacrylate, typically in granular form, which may be confined inside, or within a coating of, cellulose or other similar materials.

The support layer 12a has opposite side flaps 17a folded over the surface 13a, so that some absorbent fibres 20 are trapped between the surface 13a and the respective side flaps 17a.

The lower layer 3 and the upper layer 4 have a width substantially equal and greater than the width of the absorbent core 10a.

The absorbent pad 2 is produced starting from a support layer strip 12 by the apparatus of FIG. 7.

The support layer strip 12 has a deposition surface 13 destined to receive the absorbent fibres 20 and a service surface 15 opposite the deposition surface 13.

The apparatus 100 comprises a preparation station 110, a deposition station 120 arranged downstream of the preparation station 110 along an advancing direction A of the support layer strip 12 and a forming station 130 arranged downstream of the deposition station 120 along the advancing direction A.

In accordance with the present invention, the support layer strip 12 is initially supplied to the preparation station 110, from which it is then supplied to the deposition station 120, from which it is then supplied to the forming station 130.

In particular, the support layer strip 12 is supplied to the preparation station 110 with its opposite longitudinal flaps 17 arranged on the same plane of a central longitudinal portion 19 of the support layer strip 12, i.e. with the longitudinal flaps 17 arranged in what in this description is indicated as “initial position”.

The support layer strip 12 is then supplied from the preparation station 110 to the deposition station 120 and from the latter to the forming station 130 with its opposite longitudinal flaps 17 folded over the service surface 15, i.e. with the longitudinal flaps 17 arranged in what in this description is indicated as “intermediate position”.

The support layer strip 12 subsequently exits the forming station 130 with its opposite longitudinal flaps 17 folded over the deposition surface 13, i.e. with the longitudinal flaps 17 arranged in what in this description is indicated as the “final position”.

The preparation station 110 comprises folding members 112 configured to continuously fold the opposite longitudinal flaps 17 of the support layer strip 12 over the service surface 15 while the support layer strip 12 passes through the preparation station 110 along an advancing path A1.

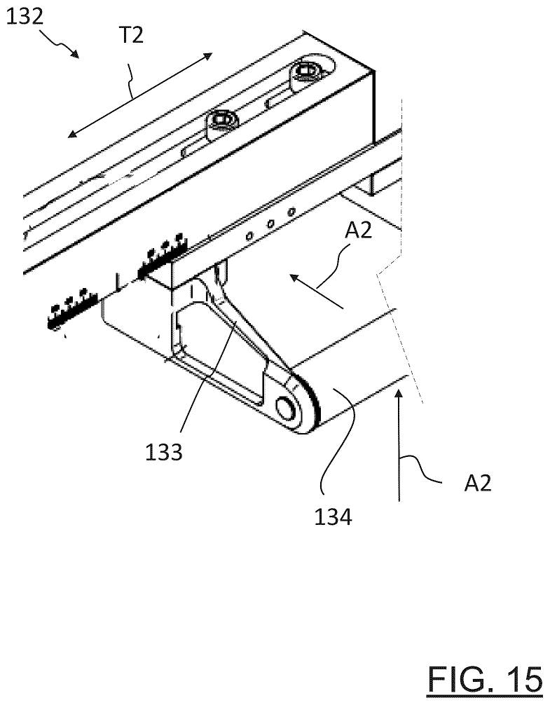

The folding members 112 may be of conventional type. For example, they may comprise suitably shaped and oriented folding elements, similar to those illustrated in FIG. 15 and described below.

The folding members 112 are movable along a transverse direction T with respect to the advancing path A1, so as to be able to be arranged from time to time in the desired position.

The preparation station 110 further comprises perforating members 114 configured to perforate the support layer strip 12 along its entire width while the support layer strip 12 passes through the preparation station 110 along the advancing path A1.

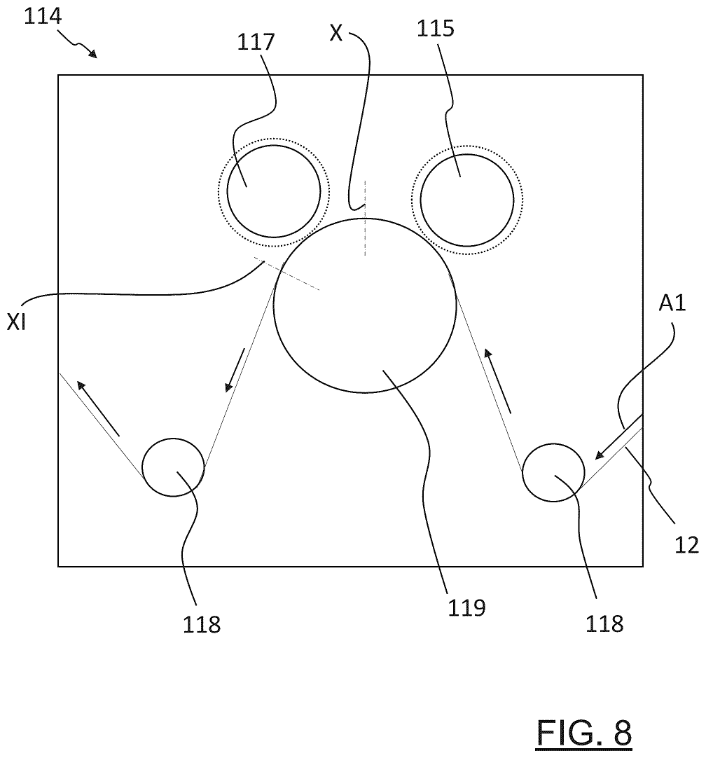

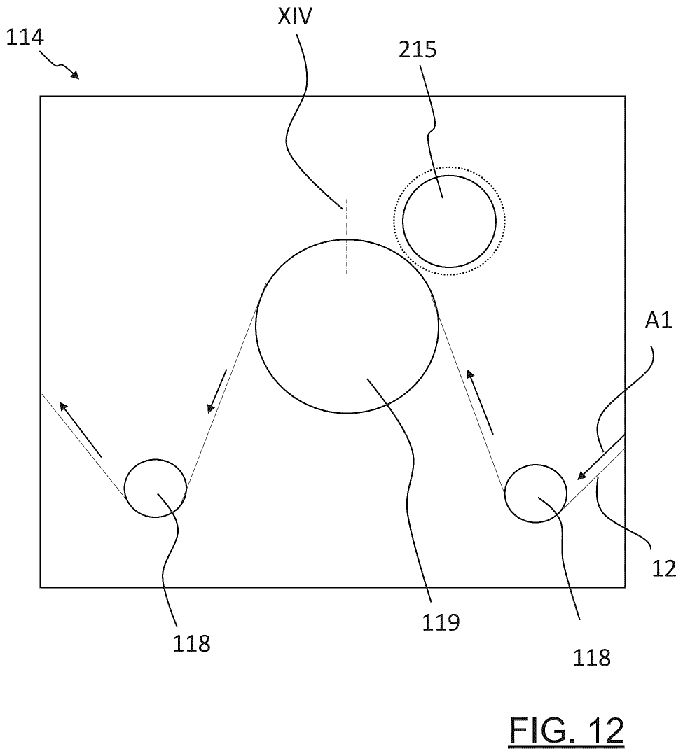

Two alternative embodiments of the perforating members 114 are illustrated in FIGS. 8 and 12.

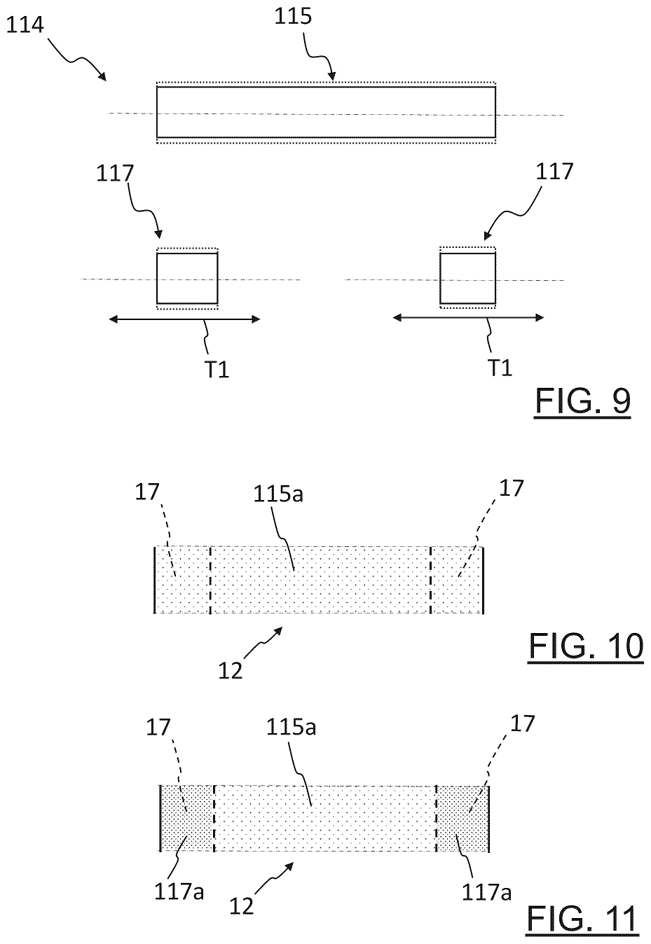

In the embodiment illustrated in FIGS. 8 and 9, the perforating members 114 comprise a first roller 115 configured to make a plurality of first holes 115a on the entire support layer strip 12.

The first roller 115 is arranged downstream of the folding members 112 along the advancing path A1.

The perforating members 114 further comprise a pair of second rollers 117 configured to each make a plurality of respective second holes 117a at a respective longitudinal flap 17 of the support layer strip 12 after this longitudinal flap 17 has been folded over the service surface 15.

The pair of second rollers 117 is arranged downstream of the folding members 112 along the advancing path A1.

In the non-limiting example of FIGS. 8 and 9, the second rollers 117 are aligned and arranged downstream of the first roller 115 along the advancing path A1. Alternatively, the second rollers 117 could be arranged upstream of the first roller 115 along the advancing path A1.

The second rollers 117 are movable along a transverse direction T1 orthogonal to the advancing path A1 so as to be able to be arranged at the longitudinal flaps 17.

The first roller 115 and the second rollers 117 are substantially tangent to a counter roller 119 on which the support layer strip 12 is arranged during its movement along the advancing path A1 and at which the first and second holes 115a, 117a are made.

The support layer strip 12 is tensioned on the counter roller 119 through a pair of return rollers 118, located upstream and downstream of the counter roller 119 along the advancing path A1.

FIG. 10 is a view of a portion of the support layer strip 12 taken downstream of the first roller 115 along the advancing path A1, in which the first holes 115a are visible on the entire support layer strip 12.

FIG. 11 is a view of a portion of the support layer strip 12 taken downstream of the second rollers 117 along the advancing path A1, in which also the second holes 117a are visible at the longitudinal flaps 17.

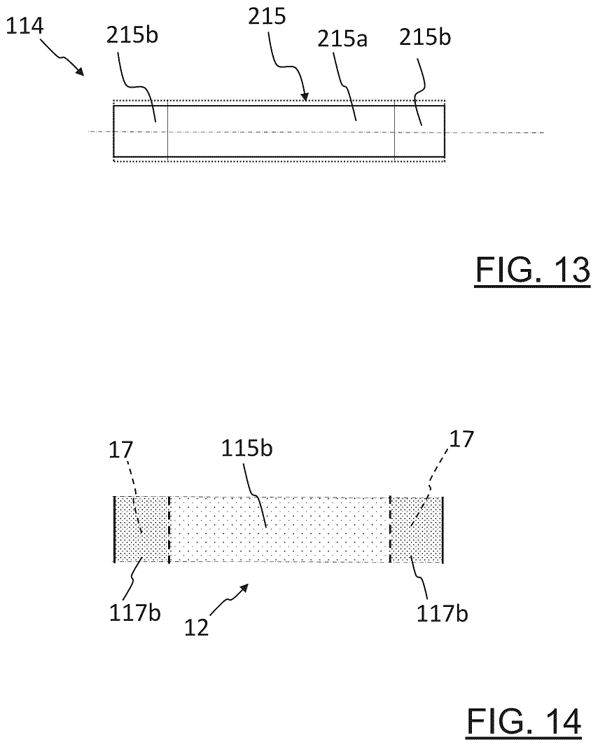

The embodiment illustrated in FIGS. 12 and 13 differs from that illustrated in FIGS. 8 and 9 only in that the perforating members 114 in this case comprise a single roller 215, in turn comprising a central portion 215a configured to make a plurality of first holes 115b on the central longitudinal portion 19 of the support layer strip 12 and opposite end portions 215b configured to each make a plurality of respective second holes 117b at a respective longitudinal flap 17 of the support layer strip 12 after this longitudinal flap 17 has been folded over the service surface 15.

The roller 215 is arranged downstream of the folding members 112 along the advancing path A1.

FIG. 14 is a view of a portion of the support layer strip 12 taken downstream of the roller 215 along the advancing path A1, in which the first holes 115b and the second holes 117b are visible.

Preferably, in both embodiments of the perforating members 114 described above, the plurality of second holes 117a, 117b differs from the plurality of first holes 115a, 115b in at least one of the following parameters: size of the holes and number of holes for the same area.

In particular, the second holes 117a, 117b define at the longitudinal flaps 17 folded over the service surface 15 a density of air passage openings greater than that at the portion of support layer strip 12 in which only the first holes 115a, 115b are made.

The deposition station 120 comprises a deposition device 122 configured to deposit the absorbent fibres 20 on the deposition surface 13.

Thus, when the support layer strip 12 passes through the deposition station 120, the deposition surface 13 faces the deposition device 122.

The deposition station 120 also comprises a conveyor belt 124 configured to support the support layer strip 12 and supply it along the advancing direction A. The conveyor belt 124 has a width greater than that of the support layer strip 12 and is arranged below the deposition device 122. Further, the conveyor belt 124 is perforated. In FIG. 7 the support layer strip 12 is voluntarily illustrated raised with respect to the conveyor belt 124 to clearly illustrate the support layer strip 12 above the conveyor belt 124. In reality, the support layer strip 12 rests on the conveyor belt 124.

The deposition device 122 generates an air flow that facilitates the deposition of the absorbent fibres 20 on the deposition surface 13. The air flow passes through the support layer strip 12 and the conveyor belt 124.

The deposition of the absorbent fibres 20 therefore takes place by fall from the deposition device 122 and by the thrust action exerted by the aforesaid air flow.

The absorbent fibres pass through an opening 122a defined in a lower surface of the deposition device 122. This opening 122a preferably has a width greater than that of the support layer strip 12. As shown in FIG. 3, the absorbent fibres 20 that do not fall onto the support layer strip 12 (herein referred to as “excess fibres”) are sucked in by two suction nozzles 128 arranged above the conveyor belt 124 at opposite sides with respect to the latter.

The absorbent fibres 20 are deposited on the central longitudinal portion 19 of the support layer strip 12. The central longitudinal portion 19 has opposite longitudinal edges 19a, at which the longitudinal flaps 17 are folded over (FIGS. 2 and 5). Thus, in the deposition station 120 the deposition surface 13 corresponds to the upper surface of the central longitudinal portion 19 of the support layer strip 12.

Two air blowers 125 are arranged downstream of the deposition device 122, with reference to the advancing direction A and at a downstream end portion 124a of the conveyor belt 124 with reference to a movement direction M thereof. The two air blowers 125 are positioned along a transverse direction with respect to the movement direction M, each near a respective side of the conveyor belt 124. Each air blower 125 is configured to generate a concentrated jet of air that laps the opposite longitudinal edges 19a and allows to clearly and precisely separate the excess absorbent fibres 20, i.e. those that following the fall are at least partially in an external position with respect to these longitudinal edges 19a, from the absorbent fibres 20 that instead are entirely deposited on the central longitudinal portion 19, and therefore on the deposition surface 13.

The air blowers 125 are movable along the transverse direction T orthogonal to the advancing direction A so as to be able to be arranged at the longitudinal edges 19a.

The forming station 130 comprises folding members 132 configured to continuously fold the opposite longitudinal flaps 17 of the support layer strip 12 over the deposition surface 13 while the support layer strip 12 passes through the forming station 130 along an advancing path A2.

### The folding members 132 may be of conventional type.

By way of non-limiting example, the folding members 132 may be of the type illustrated in FIG. 15. They comprise folding elements 133 (FIG. 15 shows only one folding element 133) intended to come into contact with the longitudinal flaps 17 of the support layer strip 12 and fold these longitudinal flaps 17 over the deposition surface 13 while the support layer strip 12 passes through the forming station 130 along the advancing path A2 and after the support layer strip 12, supported by a counter roller 134 located upstream of the folding elements 133 along the advancing path A2, is oriented so as to interfere with the folding elements 133.

The folding members 132 are movable along a transverse direction T2 with respect to the advancing path A2 so as to be able to be arranged from time to time in the desired position.

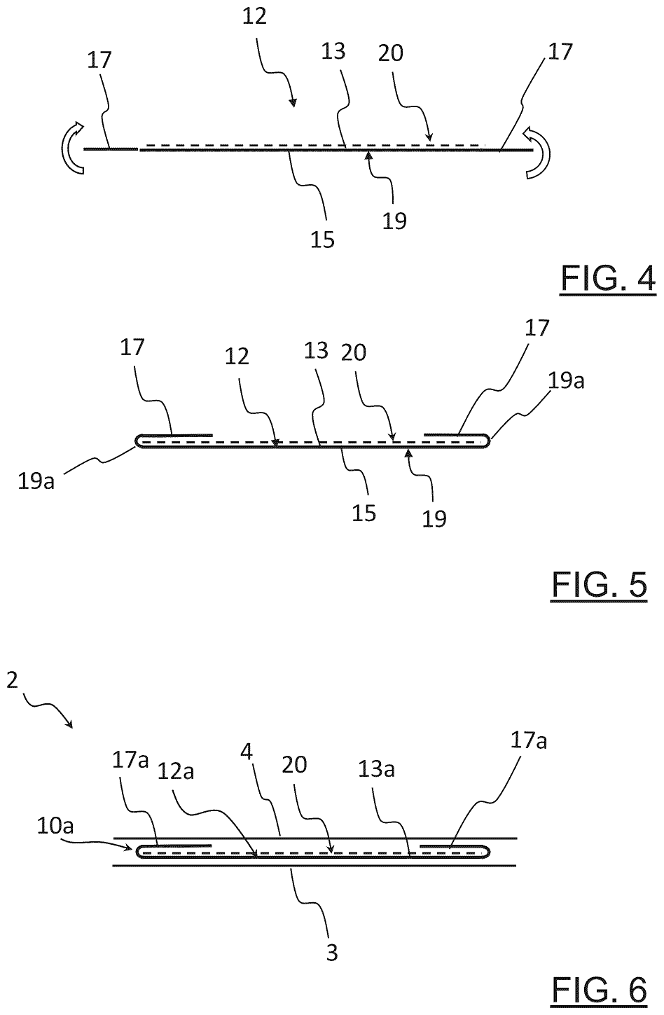

FIGS. 4 and 5 show two operating configurations of the support layer strip 12 assumed in sequence in the forming station 130, and then after the absorbent fibres 20 have been deposited on the deposition surface 13.

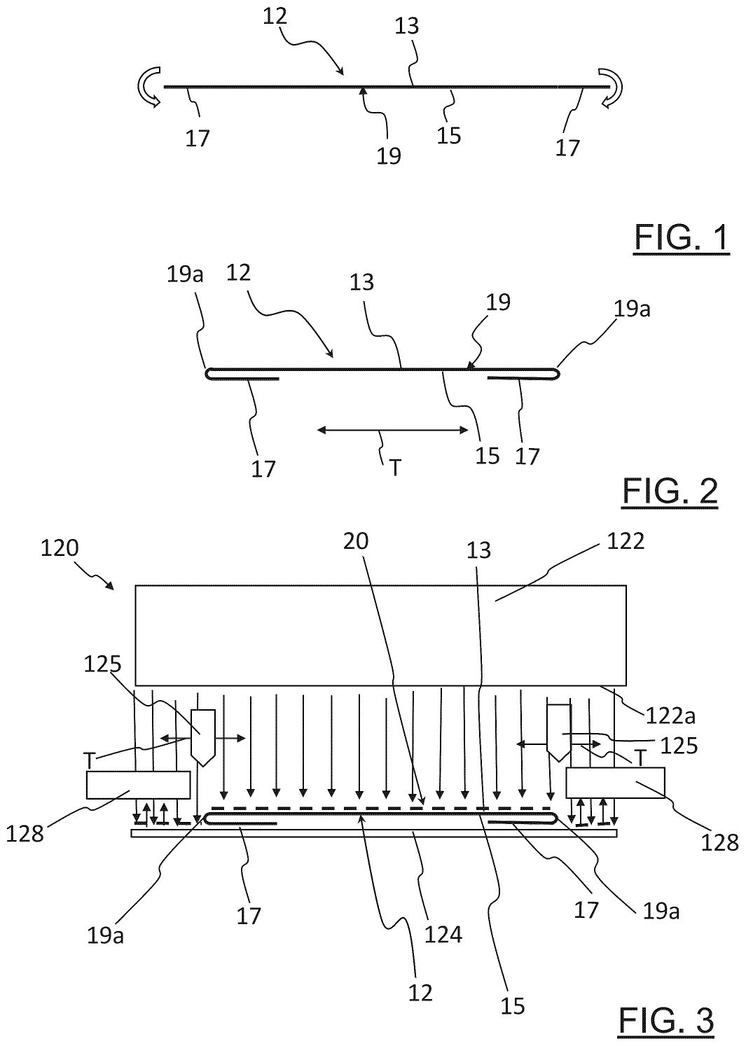

The operating configuration of FIG. 4 is assumed after the folding members 132 have brought back the longitudinal flaps 17 on the same plane of the central longitudinal portion 19, i.e. into their initial position, illustrated in FIG. 1.

The operating configuration of FIG. 5 is assumed after the folding members 132 have folded the longitudinal flaps 17 over the central longitudinal portion 19, i.e. in their final position. In this final position the longitudinal flaps 17 cover the absorbent fibres 20 deposited near the opposite longitudinal edges 19a.

The apparatus 100 further comprises, downstream of the forming station 130 along an advancing direction A3 of the support layer strip 12 (which is in the operating configuration of FIG. 5), an application station (not illustrated because of conventional type) configured to apply the strip of lower layer 3 and the strip of upper layer 4 below and above the support layer strip 12, respectively, after the longitudinal flaps 17 have been folded over the deposition surface 13.

Obviously, a person skilled in the art, in order to satisfy specific and contingent needs, can make numerous modifications and variations to the invention described above while remaining within the scope of protection defined by the following claims.

1. An apparatus for producing absorbent bed pads, or absorbent pet mats, of a type comprising an absorbent core comprising a support layer and a plurality of absorbent fibres arranged on a first surface of said support layer, said apparatus comprising:
a preparation station comprising first folding members configured to fold opposite longitudinal flaps of a support layer strip over a service surface of the support layer strip;
a deposition station arranged downstream of the preparation station along an advancing direction (A) of said support layer strip and comprising a deposition device configured to deposit said plurality of absorbent fibres on a deposition surface of said support layer strip opposite said service surface;
a forming station arranged downstream of the deposition station along said advancing direction (A) and comprising second folding members configured to fold the opposite longitudinal flaps of the support layer strip over said deposition surface.

2. The apparatus according to claim 1, wherein the first folding members are configured to continuously fold the opposite longitudinal flaps of the support layer strip over said service surface while the support layer strip passes through the preparation station.

3. The apparatus according to claim 1, wherein the first folding members are movable along a transverse direction (T) with respect to a first advancing path of the support layer strip in the preparation station.

4. The apparatus according to claim 1, wherein the second folding members are configured to continuously fold the opposite longitudinal flaps of the support layer strip onto said deposition surface while the support layer strip passes through the forming station.

5. The apparatus according to claim 4, wherein the second folding members are movable along a transverse direction (T) with respect to a second advancing path of the support layer strip in the forming station.

6. The apparatus according to claim 1, wherein the preparation station comprises perforating members configured to perforate the support layer strip along its entire width while the support layer strip passes through the preparation station.

7. A method for producing absorbent bed pads, or absorbent pet mats, of a type comprising an absorbent core comprising a support layer and a plurality of absorbent fibres arranged on a first surface of said support layer, said method comprising in sequence:
folding opposite longitudinal flaps of a support layer strip over a service surface of the support layer strip;
depositing said plurality of absorbent fibres on a deposition surface of said support layer strip opposite said service surface;
folding the opposite longitudinal flaps of the support layer strip over said deposition surface.

8. The method according to claim 7, wherein the opposite longitudinal flaps of the support layer strip are continuously folded over said service surface while the support layer strip is moved along a first advancing path.

9. The method according to claim 7, wherein the opposite longitudinal flaps of the support layer strip are continuously folded over said deposition surface while the support layer strip is moved along a second advancing path.

10. The method according to claim 7, comprising, prior to depositing said plurality of absorbent fibres on said deposition surface, perforating the support layer strip along its entire width.
